(19) United States
(12) Patent Application Publication
(10) Pub. No.: US 2016/0287339 A1
(43) Pub. Date: Oct. 6, 2016

(54) METHOD FOR MANUFACTURING A THREE-DIMENSIONAL ANATOMICAL STRUCTURE

(71) Applicant: Universiti Malaya, Kuala Lumpur (MY)

(72) Inventors: Zainal Ariff Bin Abdul Rahman, KUALA LUMPUR (MY); Vickneswaran A/L Mathaneswaran, KUALA LUMPUR (MY); Alwin Kumar Rathinam, KUALA LUMPUR (MY); Yuwaraj Kumar A/L Balakrishnan, KUALA LUMPUR (MY); Su Tung Tan, KUALA LUMPUR (MY)

(21) Appl. No.: 15/100,324

(22) PCT Filed: Apr. 21, 2014
(86) PCT No.: PCT/MY14/00064

§ 371 Date: May 30, 2016

(30) Foreign Application Priority Data
Apr. 30, 2013 (MY) PI 2013700700

(51) Int. Cl. A61B 34/10; G05B 19/4099; A61F 2/02; A61B 6/03; A61B 8/00

(52) U.S. Cl. CPC A61B 34/10; A61B 6/03; A61B 8/00; A61F 2/02; G05B 19/4099; A61B 2034/105; A61B 2034/108; G05B 2219/49023

(57) ABSTRACT

A method for manufacturing a three-dimensional anatomical structure comprising the steps of obtaining a plurality of medical images containing at least one anatomical region of interest, wherein each anatomical region of interest is defined based on a grey level value on the medical images; segmenting each medical image based on the anatomical region of interest to obtain the grey level value; converting the grey level values of the respective segmented medical images into vector data; interpolating the vector data of each segmented medical image to form a three-dimensional data; and producing a three-dimensional anatomical structure of the region of interest from the three-dimensional data.

### FIELD OF INVENTION

This invention relates to a method for manufacturing a three-dimensional (3D) anatomical structure. In more particular, this invention relates to a method for manufacturing a 3D anatomical structure based on the grey level value of the medical images.

### BACKGROUND OF THE INVENTION

Creating artificial organs through 3D modeling is a common and important practice in the medical industry. The produced 3D models must be as similar to patient's body parts as possible to enable physicians, surgeons and radiologists to visualize, rehearse, diagnose or plan for surgery and treatment procedure of a patient.

Several processes such as milling, drilling or turning have been commonly used to produce 3D models, nevertheless, these processes are relatively wasteful as materials from the work piece are cut off to form the desired models. Apart from that, moulding or casting processes are used to produce 3D models by solidifying production materials in moulds fabricated according to shapes and sizes of the desired body parts. However, fabrication of merely the moulds require abundant of time, especially for moulds with high complexity and large size.

There are some patent technologies over the prior arts relating to methods to produce 3D models. As disclosed in a U.S. Pat. No. 5,932,059(A), a method for producing a 3D object is generated by solidification of individual layers of an object in an inner core region and an outer shell region using electromagnetic radiation. However, the invention focuses on the production of 3D object by calculations of merely coordinates of the object to control the electromagnetic radiation.

In U.S. Pat. No. 20100134487(A1), a method to construct a 3D human face model is disclosed. The method comprises entering a two-dimensional (2D) image in a system to obtain a plurality of face feature points and build a 3D expression face model. This invention suggested the application in facial animation and recognition and merely produces the outer surface of faces expressions.

In another U.S. Pat. No. 20100054572(A1), an image analysis method is disclosed. Suitable image, includes magnetic resonance imaging (MRI), are obtained in plurality of planes to generate a 3D data volume, where the 3D data volume can be further derived to produce an implant. However, the generated 3D data volumes are combined with each other to form an isotropic or near-isotropic image volume.

A journal article entitled ‘Stereolithographic Biomodeling of Congenital Heart Disease by Multi-slice Computed Tomography Imaging’ by Isao Shiraishi, 2006, has disclosed a technique to produce a 3D volumetric model by obtaining data acquisition from multislice computed tomography (CT) and utilizing the data to guide an ultraviolet laser beam to generate a 3D model. Another journal entitled ‘Use of 3D Geometry Modeling of Osteochondrosis-like Iatrogenic Lesions as a Template for Press-and-Fit Scaffold Seeded with Mesenchymal Stem Cells’ by P. Krupa, et al., 2007 also discloses a process for generating 3D model using CT images. Rapid prototyping is used to produce the 3D model by printing glue on a plaster layer. However, the 3D images disclosed by these prior arts are merely generated by image analysis software by defining the volume of interest.

Because of the lack of accurate radiological information, the physicians were not able to achieve good alignment and hence likely to cause failure of surgeries. Multiple surgeries would result higher risk to the patient, as well as more costs and significant additional recovery times. Therefore, it is highly desirable for the present invention to produce informative 3D models from 2D images that enables accurate surgical planning, rehearsal and training.

### SUMMARY OF INVENTION

One of the objects of the present invention is to construct a physical 3D anatomical structure of a region of interest from a plurality of medical images using a reliable and efficient method.

Another object of the present invention is to assess the relationships of soft tissues, airway, skin, bones and joints in a 3D anatomical structure, thus providing a 3D structure having regions similar to the actual tissue, airway, skin, bone, joint or the combination thereof.

It is yet another object of the present invention to provide an informative 3D anatomical structure of a specific body part to enable accurate surgical planning, rehearsals and training.

A further object of the present invention is to provide a 3D anatomical structure of a specific anatomical region with a high similarity to the actual anatomical region of interest of a patient.

At least one of the proceeding objects is met, in whole or in part, by the present invention, in which the preferred embodiment of the present invention describes a method for manufacturing a 3D anatomical structure comprising the steps of obtaining a plurality of medical images containing at least one anatomical region of interest, wherein each anatomical region of interest is defined based on a grey level value on the medical images; segmenting each medical image based on the anatomical region of interest to obtain the grey level value; converting the grey level values of the respective segmented medical images into vector data; interpolating the vector data of each segmented medical image to form a 3D data; and producing a 3D anatomical structure of the region of interest from the three-dimensional data.

One of the preferred embodiment of the present invention discloses that the step of forming the 3D data is conducted by using a Marching cube algorithm, Delaunay's triangulation algorithm or a combination thereof.

In one of the embodiment of the present invention, the 3D anatomical structure is made from different materials, such as plastic, plaster, steel, alloy or any two or more combinations thereof, depending on the anatomical region of interest.

Another embodiment of the present invention is that the anatomical region of interest is bone, skin, organ, tumour or a combination of any two or more thereof.

Still another embodiment of the present invention is that the medical images are X-ray images, computed tomography images, magnetic resonance images, ultrasound images, positron emission tomography images or single-photon emission computed tomography images.

A further embodiment of the present invention discloses that the step of producing the 3D anatomical structure is by using a rapid additive manufacturing machine.

A particular embodiment of the present invention discloses that the rapid additive manufacturing machine includes techniques of layered manufacturing, direct digital manufacturing, laser processing, electron beam melting, aerosol jetting, semi-solid free-form fabrication or a combination of any two or more thereof.

Another further embodiment of the present invention discloses a computer program or software for producing a 3D data from medical images to manufacture a 3D anatomical structure comprising the steps of receiving input of a plurality of medical images containing at least one anatomical region of interest, wherein each anatomical region of interest is defined based on a grey level value on the medical images; segmenting each medical image based on the anatomical region of interest to obtain the grey level value; converting the grey level values of the respective segmented medical images into vector data; and interpolating the vector data of each segmented medical image to form a 3D data for the manufacture of a 3D anatomical structure of the region of interest.

The present preferred embodiments of the invention consists of novel features and a combination of parts hereinafter fully described and illustrated in the accompanying drawings and particularly pointed out in the appended claims; it being understood that various changes in the details may be effected by those skilled in the arts but without departing from the scope of the invention or sacrificing any of the advantages of the present invention.

### DETAILED DESCRIPTION OF THE INVENTION

Hereinafter, the invention shall be described according to the preferred embodiments of the present invention and by referring to the accompanying description and drawings. However, it is to be understood that limiting the description to the preferred embodiments of the invention and to the drawings is merely to facilitate discussion of the present invention and it is envisioned that those skilled in the art may devise various modifications without departing from the scope of the appended claim.

The present invention discloses an innovative method for manufacturing a 3D anatomical structure. In more particular, this invention relates to a method for manufacturing a 3D anatomical structure based on the grey level value of the medical images.

One of the preferred embodiments of the present invention discloses that, a plurality of medical images can be obtained from a patient or any biological organism, and each of these medical images is segmented to obtain at least one region of interest. The plurality of medical images, or may otherwise be referred to as medical scan images, are rendered together to produce an actual 3D image of the region of interest, instead of imagining a virtual 3D model of the region of interest. It is to be understood that, the medical images are images of the transverse, coronal, or sagittal planes of a patient or biological organism and the planes depend on a diagnostic task.

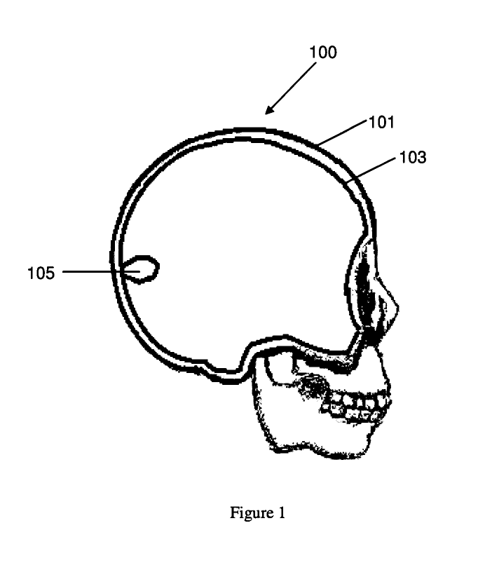

Referring to FIG. 1, a medical image of a human's skull is shown. The medical image shows a plurality of regions having different grey level values. The medical images have a plurality of volumetric pixels and each pixel correspondences to a grey level value. A void region (103) is shown to have the darkest shade, with a grey level value of 0. The anatomical regions (105) are shown to have lighter shades with grey level values in a range of 1 to 255 for a 8-bit per pixel image. The medical images with high contrast of resolution are able to distinguish the differences between different anatomical regions that differ in physical density, usually by less than 1%. Through the step of segmenting the medical images, the void region (103) is preferred to be eliminated. Depending on the region of interest, a preferred grey level value is selected and converted to a vector data. Upon segmenting the region of interest, any noise, artifacts or undesired regions are eliminated or reduced.

According to an embodiment of the present invention, the grey level value of each region of interest ranges from 0-255 for images with 8-bits per pixel. The medical images show that each pixel has an intensity of grey shade. The weakest intensity is black, the strongest intensity is white and there are many shades of grey in between. For medical images with colour scales, the images are preferred to be converted to greyscales images as vectorization of coloured images produces poor results. The medical images are preferred to be analyzed in a computing device, and the intensity of the grey shades is computed through the grey level values that can be stored in binary or quantized forms. The values are converted to vector data by a mathematical equation. The mathematical equation is preferred to be a linear equation. The vector data is preferred to be stored in a polygon (PLY) file format as it is simple, fast in saving and loading as well as easy to be implemented for a wide range of computer programmes.

According to another embodiment of the present invention, the 3D data is subjected to a rapid additive manufacturing machine where layers of material are added upon one another to form the desired 3D anatomical structure. The rapid additive manufacturing machine comprises techniques including layered manufacturing, direct digital manufacturing, laser processing, electron beam melting, aerosol jetting or semi-solid free-form fabrication. The 3D data enables the rapid additive manufacturing machine to sequentially build up many thin layers upon one another to obtain the anatomical structure. No additional tooling or moulds are required and thus, the process exceptionally reduces fabrication time of the anatomical structure.

A particular embodiment of the present invention discloses that the step of forming the three-dimensional data is by using a Marching cube algorithm, Delaunay's triangulation algorithm or a combination thereof. Marching cube algorithm, Delaunay's triangulation algorithm or the combination thereof are preferred to be used due to its isotropic ability to expand pixels of the vector data in a single direction. The pixels are interpolated to form connecting series of pixel pairs. Printing of the 3D data generates a 3D anatomical structure with a continuous and smooth surface. The 3D data comprises vector data in forms of arcs and lines that are geometrically and mathematically associated as well as stored as a series of pixel pairs.

Based on the embodiment of the present invention, it is to be clearly understood that the medical images may be any images that are capable of capturing anatomical regions of a patient or biological organism. The medical images can be X-ray images, computed tomography images, magnetic resonance images or any other medical images.

In a further embodiment of the present invention, the three-dimensional anatomical structure is made from different material depending on the region of interest. The 3D anatomical structure produced from the methods as described in the embodiments of the present invention has high similarity to a real anatomical region. The 3D structure can be produced to have different layers or regions of tissues such as bones, skin and soft tissues, resembling the texture or colour of the actual anatomical region.

By way of manufacturing the 3D anatomical structure, several advantages can be obtained. By using a 3D anatomical structure, it resembles to an anatomical region of an actual patient organism. A surgeon is able to perform a rehearsal surgery prior to the actual operation by enabling the surgeon to mark, cut and operate the 3D structure as well as to provide a clear and accurate depiction. Minutes or hours of operating time can be reduced by careful preparation using the 3D structure. Besides, the present invention enables a surgeon to communicate with the patient about an upcoming surgery diagnosis through the patient's own 3D anatomical structure instead of explaining through a textbook drawing or a generic anatomical model. This also enables the patient to understand his or her diagnosis, health conditions and benefits of treatments. On the other hand, surgical trainees are able to learn and perform surgery similar to the actual surgery. For surgical trainees without prior surgical experience, the 3D anatomical structure provides an intensive training that will decrease the time taken to complete an actual surgery, increase accuracy, and decrease errors.

Another further embodiment of the present invention discloses a computer program or software for producing a 3D data from medical images to manufacture a 3D anatomical structure comprising the steps of receiving input of a plurality of medical images containing at least one anatomical region of interest, wherein each anatomical region of interest is defined based on a grey level value on the medical images; segmenting each medical image based on the anatomical region of interest to obtain the grey level value; converting the grey level values of the respective segmented medical images into vector data; and interpolating the vector data of each segmented medical image to form a 3D data for the manufacture of a 3D anatomical structure of the region of interest. This software can be installed in a computer-readable medium, and be used together with the rapid additive manufacturing system for the manufacturing of the 3D anatomical structure, based on the 3D data generated by the software.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

1-8. (canceled)

9. A method for manufacturing a three-dimensional anatomical structure, comprising the steps of:
obtaining a plurality of medical images containing at least one anatomical region of interest including bone, skin, organ, tumour or a combination of any two or more thereof, wherein each anatomical region of interest is defined based on a grey level value on the medical images;
segmenting each medical image based on the anatomical region of interest to obtain the grey level value;
converting the grey level values of the respective segmented medical images into vector data;
interpolating the vector data of each segmented medical image to form a three-dimensional data formed by using a Marching cube algorithm, Delaunay's triangulation algorithm or a combination thereof; and
producing a three-dimensional anatomical structure of the region of interest from the three-dimensional data, in which the three-dimensional anatomical structure is made from plastic, plaster, steel, alloy or any two or more combinations thereof depending on the anatomical region of interest by using a rapid additive manufacturing technique including rapid layered manufacturing, direct digital manufacturing, laser processing, electron beam melting, aerosol jetting, inkjet, semi-solid free-form fabrication or a combination of two or more thereof.

10. The method according to claim 9, wherein the medical images are X-ray images, computed tomography images, magnetic resonance images, ultrasound images, positron emission tomography images or single-photon emission computed tomography images.

11. A system for producing three-dimensional data from medical images to manufacture a three-dimensional anatomical structure, comprising:
a system for receiving input of a plurality of medical images containing at least one anatomical region of interest including bone, skin, organ, tumour or a combination of any two or more thereof, wherein each anatomical region of interest is defined based on a grey level value on the medical images;
a system for segmenting each medical image based on the anatomical region of interest to obtain the grey level value;
a system for converting the grey level values of the respective segmented medical images into vector data formed by using a Marching cube algorithm, Delaunay's triangulation algorithm or a combination thereof; and
a system for interpolating the vector data of each segmented medical image to form a three-dimensional data for the manufacture of a three-dimensional anatomical structure of the region of interest, in which the three-dimensional anatomical structure is made from plastic, plaster, steel, alloy or any two or more combinations thereof depending on the anatomical region of interest by using a rapid additive manufacturing technique including rapid layered manufacturing, direct digital manufacturing, laser processing, electron beam melting, aerosol jetting, inkjet, semi-solid free-form fabrication or a combination of two or more thereof.
